(19) United States
(12) Patent Application Publication
(10) Pub. No.: US 2004/0111110 A1
(43) Pub. Date: Jun. 10, 2004

(54) Temporary peripheral blood filtration methods to reduce or prevent post-surgery related cognitive dysfunction

(76) Inventor: Richard J. Melker, Gainesville, FL (US)

Correspondence Address: BEUSSE BROWNLEE WOLTER MORA & MAIRE, 390 N. ORANGE AVENUE, SUITE 2500, ORLANDO, FL, 32801, US

(21) Appl. No.: 10/310,705

(22) Filed: Dec. 5, 2002

(51) Int. Cl. A61M 29/00

(52) U.S. Cl. 606/200; 128/898

(57) ABSTRACT

Methods are disclosed and exemplified in which a temporary intravascular filter is inserted into a vessel to trap emboli that may be released during or after a surgical procedure. Such filtering removes emboli in order to reduce or eliminate post-surgical cognitive dysfunction. In particular, the methods are directed to dealing with surgeries conducted on the limbs, and filtering in a vein that returns blood from the limb undergoing surgery. More particularly, the methods are directed to filtration during a surgery on a limb in which blood flow is restricted to the surgical field, in which cases a greater quantity of emboli are expected to be generated as a result of the surgical procedure.

### FIELD OF THE INVENTION

The present invention is directed to methods of filtration of blood in situ in a surgical patient. More particularly, the present invention discloses methods of filtering blood passing from a surgical site, where such filtering removes emboli in order to reduce or eliminate post-surgical cognitive dysfunction.

### BACKGROUND OF THE INVENTION

Filters for use in arteries, and also in veins, are known and in common use for a variety of specific purposes. Frequently, filters are used to capture material freed during surgery, in particular during cardiac surgery (see, for example, U.S. Pat. No. 6235045), and during arterial vascular surgery (see, for example, U.S. Pat. No. 6346116). During such procedures, it is generally recognized that there is a risk of emboli being dislodged by the surgical procedure, and such emboli migrating in the arterial blood vessels and ultimately blocking blood flow when the caliber of arteries are less than the diameter of the emboli. Emboli as used herein may consist of clotted blood or blood elements, atheromatous plague, bone fragments, fat, marrow elements or combinations of these elements. If the emboli migrate to the brain they may result in a stroke. If the emboli migrate to the arteries supplying the heart, they may result in myocardial infarction, and if they migrate to peripheral vessels they may lead to ischemia and ultimately loss of the extremity.

Generally, filters are not employed during a surgery that may cause emboli to traverse the venous side of the circulation, as it is accepted that these small emboli lodge in the lung and cause little disturbance. Larger emboli, especially from the lower extremities or pelvis may block the main pulmonary artery or a distal pulmonary artery segment leading to significant morbidity or death.

For arterial emboli, for instance, U.S. Pat. No. 4,873,978 teaches a catheter device “for capturing and retrieving emboli from a blood vessel,” where the device is comprised of an elongate catheter body, a strainer at the distal end of the body, and a means workable from the proximate end of the body to “shift[ing] the tines . . . between the open and the closed configuration” (from claim 1). The specification describes uses that include the capture of plaque or thrombi during an angioplastic procedure, such as balloon angioplasty, laser angioplasty, suction angioplasty, and atherectomy (see col. 1, lines 13-27). This patent is representative of numerous patents in the field that claim specific combinations of structures that open a filter once a catheter is inserted to a desired positions in a vessel, and that close the filter prior to removal by a linkage to a control mechanism.

U.S. Pat. No. 6,051,015 claims a method of protecting a patient comprising steps that include introducing a tubular member into the patient's vessel where the tubular member has a lumen from the proximal to distal ends; inserting and advancing a filter element through the lumen; deploying the filter; removing the filter; and inserting a second filter. Claim 14 is directed to a “method of temporarily filtering embolic material from the blood in a blood vessel, . . . wherein embolic material is generated and filtered before the filter is removed from the vessel. The object is to remove embolic material with the filters, such as during extended heart surgeries. Also, in the '015 patent and its continuation application, issued May 1, 2001 as U.S. Pat. No. 6,224,620, it is noted that a new filter may be inserted “prior to any action which may substantially increase the risk of further embolic material breaking loose, such as when the aorta is unclamped” (col. 9, lines 10-13 or '620 patent).

U.S. Pat. No. 6,383,206 is directed to a system and method claims for “capturing embolic material which may be released into a blood vessel during a therapeutic interventional procedure.” A therapeutic intervention, per the '206 disclosure, is focused on procedures such as balloon angioplasty, stenting, laser angioplasty or atherectomy. These are considered to be outside the scope of traditional surgery.

For venous emboli, U.S. Pat. No. 6,231,589 principally regards the use of a filter in the lumen of the inferior vena cava to prevent pulmonary embolism. The filter may be used for temporary or permanent applications. All claims are directed to a specific design of a filter assembly.

U.S. published applications 2002/002384 A1 and U.S. 2002/0026213 A1 are largely directed to specific embodiments of a filtration device placed in blood vessels to capture and remove embolic material during an interventional procedure. In most embodiments, the filtration device is deployed and positioned along, rather than at the end, of a guide wire. Advantageously, the same guide wire can be used to deploy devices such as a balloon catheter and a stent. It is stated that the use of such interventional devices may tend to cause the release of material from the lumen of the blood vessel, and the filter, being positioned downstream of such devices, is in a position to capture such emboli.

Thus, there is ample art in the field of intravascular filtration devices, particularly with specific combinations of mechanical remote control and filtration components. However, these references do not specifically recognize the utility of methods to reduce or prevent post-surgical cognitive dysfunction that may result from surgeries in general, or more particularly from arthroscopic and other surgeries on extremities, especially those that use a blood restricting apparatus, such as a tourniquet.

The inventor of the present invention and colleagues have observed that patients recovering from surgical procedures not infrequently experience some level of cognitive dysfunction. It has also been observed that the severity of such cognitive dysfunction usually, but not always, diminishes over time. Such improvement in cognitive function may be related to the neuroplasticity of the brain, as generally described in “The Mind and the Brain: Neuroplasticity and the Power of Mental Force,” Jeffrey M. Schwartz & Sharon Begley, ReganBooks, 2002, or more likely is due to anesthetic and other drugs being cleared from the body or is due to as yet unknown factors.

However, despite the possibility of recovery over time, a patient's subjective experience of cognitive dysfunction, and the limitations such condition imposes upon caretakers, increases the personal and health care costs for surgeries. Also, prospective patients who hear of or observe such side effects may be less willing to consent to surgery, fearing similar complications.

Accordingly, there is a previously unrecognized need in the art for improvement in the method of use of intravascular filtration devices to reduce or eliminate post-surgical cognitive dysfunction.

### SUMMARY

The inventor has observed that many post-surgery patients, having recovered from anesthesia, nonetheless display signs and symptoms of cognitive dysfunction. These signs and symptoms of cognitive dysfunction, which may be apparent to the patient, may persist for days, weeks, and in some cases, for months or permanently. There appears to be a higher incidence of such postsurgery cognitive dysfunction in the more elderly population of surgical patients. Cognitive dysfunction appears to be most common and severe after particular orthopedic surgeries in which a restriction, such as a tourniquet, was applied to create a relatively blood-free surgical field in an extremity, such as for knee replacement surgery.

One probable cause of cognitive dysfunction is the blockage of blood vessels in the brain by emboli. However, it is generally accepted that emboli arising in the venous circulation are “filtered” by the lungs and do not, under ordinary circumstance, enter the arterial circulation and lodge in the brain. Most clinicians assume that if cognitive dysfunction is due to venous emboli entering the arterial circulation, then it occurs only in patients with a patent foramen ovale (a congenital communication, or potential communication, between the cardiac atria) and that the emboli cross from the venous to arterial circulations in the cardiac atria, before the blood reaches the lungs. This is a rare condition and does not explain the high incidence of cognitive dysfunction seen in postoperative patients.

There is now clinical and experimental evidence that blood clots, fat, bone fragments and other embolic debris resulting from orthopedic procedures are released into the venous system and migrate to the lungs. If a tourniquet is placed before the orthopedic procedure, so that the surgery can be performed in a bloodless field, then there is a massive shower of emboli upon tourniquet release. Again, it has been generally accepted that this is not a significant problem because of the filtering capabilities of the lungs. Recent studies at the institution of the inventor suggest that significant quantities of emboli traverse the lungs and enter the arterial circulation, eventually lodging in the brain and other organs.

Thus, while tourniquets, and other means to restrict blood flow, provide a relatively “bloodless” surgical field during an operation, it is hypothesized that this can lead to a high incidence of emboli released after a period of no blood flow. Having observed the occurrence of post-surgery cognitive dysfunction, particularly in procedures involving the use of a tourniquet to restrict blood flow to the surgical field, it is now believed that:

1) a large number of emboli are released into the venous blood flow during or after a surgical procedure, particularly when a tourniquet is loosened after surgery;

2) because of the large number of emboli released, the normal protective filtering function of the lung is overwhelmed and emboli traverse the lung, enter the arterial circulation and lodge in the blood vessels of the brain; and

3) this is a cause of diminished cognitive function after a surgical procedure.

In support of this hypothesis, persons in the art have advanced the belief, for example, that myocardial infarction, deep vein thrombosis, and pulmonary embolism all involve undesirable fibrin clot formation (See U.S. Pat. No. 6,462,050, U.S. patent application Publication No. U.S.20020022604A1).

Further, blood that is stagnant, for instance in a vessel in which flow is being restricted during a surgical procedure, is more likely to clot than normally flowing blood likely due to endothelial cell damage. This damage triggers platelet aggregation and tissue thromboplastin release that, in turn, triggers the coagulation cascade, resulting a the formation of a thrombus starting at the site of the endothelial cell damage.

Without being bound the this theory, the installation of a temporary venous filter that catches these emboli provides a solution to the observed increase in post-surgical cognitive dysfunction, especially for “bloodless” surgeries performed with the aid of a tourniquet or the like. Preferably, the filter is installed for use prior to tourniquet release in an appropriate vein, such as the iliac or femoral vein and is “deployed” (expanded) just prior to tourniquet release. The filter is removed, taking the emboli with it, after sufficient blood flow has passed most or all emboli to the filter. As appropriate for the particular surgical operation and patient, a filter that has emboli in it may be replaced with a second, or subsequent, filters that continues to capture emboli.

Thus, one or more temporary venous filters are placed into use to capture and remove emboli resulting from a surgical procedure, particularly a surgical procedure in which a blood restriction apparatus, such as a tourniquet, is employed, such as to provide a blood-free surgical field. The filters are inserted in a “collapsed” state until just prior to release of the tourniquet, at which time they are deployed. Keeping the filter inn a collapsed state until needed reduces the potential of blood stagnation at the site of the filter. The invention is particularly useful to place a temporary filter in a vein receiving blood return from a limb which is undergoing a surgical procedure to reduce or eliminate post-surgical cognitive dysfunction, and is expected to have a greater relative benefit in such surgeries where a blood restriction apparatus is employed during the surgery. In general, the use of such filtration methods under a range of surgical situations results in diminished or no post-surgical cognitive dysfunction that would otherwise be brought on by emboli released during or after the surgical procedure.

### DETAILED DESCRIPTION OF THE INVENTION

Definitions

“Embolus” and “emboli” are taken to include, without being limited to, at least the following components: clotted blood or blood elements; atheromatous plague, bone fragments; fat; marrow elements; and combinations and/or associations of these elements.

“Peripheral” is taken to mean related to the extremities, i.e., the limbs (arms and legs) of a person or animal subject of a surgical procedure.

The term “patient” used herein is taken to mean mammals such as primates, including humans, sheep, horses, cattle, pigs, dogs, cats, rats, and mice.

Methods and Related Description

Surgeries to the areas of the limbs particularly afford an opportunity to provide filtration of the blood returning from the site of the surgery prior to this blood mixing with blood being returned from other limbs or sections of the torso. Also, in certain types of surgeries to the limbs, there is an opportunity to restrict blood flow to the surgical field by applying a blood restriction devices, such as, but not limited to, a tourniquet. A surgeon may prefer this in order to effect a more efficient and/or more effective surgery and/or surgical outcome.

However, without being bound to a particular theory, the use of a tourniquet, and the consequent trapping of blood and particulate matter dislodged or created (i.e., bone spicules, bone marrow elements) by the surgical procedure, for a period in the extremity, is believed to increase the frequency and/or severity of post-surgical cognitive dysfunction. While this cognitive dysfunction may resolve in many patients over a period of weeks or months, this is not a desirable surgical outcome, and may permanently diminish the cognitive functioning of some patients, particularly older patients who are at risk of other cognitive dysfunction maladies.

Further, it is believed that even without using a blood restriction device during surgery, post-surgical cognitive dysfunction may result. Thus, the examples of the methods of the present invention, as described below, are meant to apply to any surgery performed on a body extremity, whether or not a blood restriction device is applied. Also, the examples of orthopedic surgery are not meant to limit the scope of the invention only to this type of surgery on the limbs.

With regard to orthopedic surgery involving the extremities, such as knee replacement surgery, it is recognized that complications include blood clotting in the veins of the legs (Deep Venous Thrombosis, DVT) and a blood clot moving to the lungs, resulting in a Pulmonary Embolus (PE). DVT is believed to result from decreased circulation to the legs, such as during a surgery such as knee replacement. Generally, when there is little to no movement in the veins of the leg (venous stasis), blood clotting can result. Although blood thinning medication can be used to help counteract this, blood clots may nonetheless form, especially when a restriction of blood is introduced to provide a relatively bloodless field of surgery. Further, blood thinning medications are often contraindicated because while they reduce clotting, they leave the patient susceptible to severe hemorrhage, especially at the surgical site.

A PE occurs when blood clots that form in the legs or other anatomical areas detach and travel via venous circulation into the lungs. This complication is not rare and, can be life threatening. Symptoms include chest pain and shortness of breath. Other uncommon, serious complications noted in the literature include death, heart attack, stroke, , and lung infarction.

While the DVT and PE complications are generally recognized, the present invention additionally recognizes, and provides methods to deal with, a previously unappreciated complication from surgeries, particularly surgeries involving the restriction of blood flow to a region of the extremities. That complication is cognitive dysfunction. Without being bound to a particular theory, it is believed that emboli, particularly small emboli composed of blood and marrow elements, bone and cartilage that are formed or dislodge as part of the surgical procedure of preparing the bones for the joint replacement components, pass through the lungs and settle in areas of the brain, leading to localized blockage of blood supply. This is particularly more likely to occur when there is an abrupt and large efflux of emboli following the release of a blood restriction device (such as a tourniquet) and blood that has been “stagnant” (i.e., undergoing little or no movement during an surgical procedure) for a period is released.

The roles and interactions of different constituents in beneficial and in harmful clot formation are disclosed in Handin, R. I.: Bleeding and Thrombosis, In Wilson, J., et al. editors: Harrison's Principles of Internal Medicine. 12th Edition, New York, McGraw-Hill Book Co., 1991, p.350, and in Majerus, P. W. et al: Anticoagulant, Thrombolytic, and Antiplatelet Drugs. In Hardman, J. G. and Limbird, L. E., editors: Goodman & Gilman's The Pharmacological Basis of Therapeutics. 9th edition. New York, McGraw-Hill Book Co., 1996, pp. 1341-1343, which are incorporated by reference in their entireties.

As described in the several examples below, the present invention advances the art by providing methods to capture such emboli to reduce or eliminate post-surgical cognitive dysfunction related to the release of such emboli.

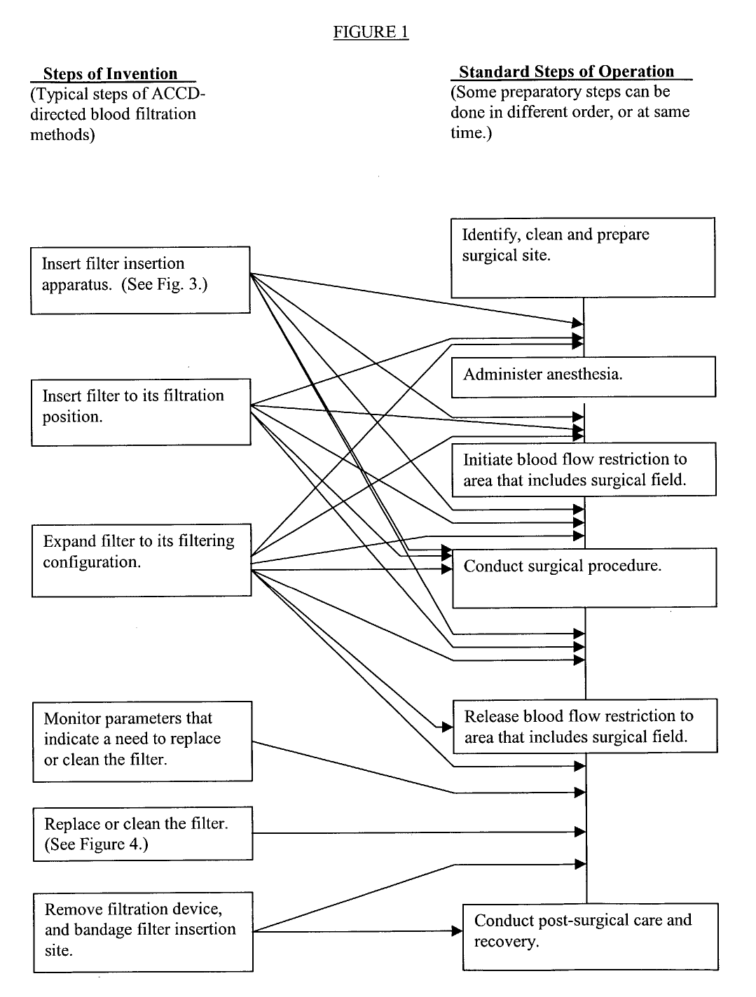

FIG. 1 provides a schematic flow diagram of standard steps in a surgical procedure and includes typical steps of anti-cognitive dysfunction-directed (“ACDD”) blood filtration methods according to the present invention. The standard steps of the surgical procedure are arranged in the right column. The typical steps of the invention are arranged in the left column. Lines ending with arrows from the steps in the left column indicate the typical points in a particular surgical procedure at or during which the respective step of the present invention's step may be conducted. As indicated by the multiple lines and arrows from a single step of the invention (left column), there is a range of alternatives for the timing of each such step in a particular surgical procedure. This will depend on a number of factors, including, but not limited to: the type of surgery; the condition of the patient; the level of advanced planning of the surgical procedure (e.g., planned vs. emergency); the availability of sufficient surgical staff; and the personal preference of the physician tasked with the filtration (in concert with the lead surgeon).

Early steps in a standard surgery include, but are not limited to: identifying, cleaning and preparing the site of surgery; and administering anesthesia. Depending on one or more of the factors above, the step (which, per below, involves sub-steps) of inserting the filter insertion apparatus (i.e., a catheter for delivery of the filter to its filtering location in a vein), and the steps of inserting the filter to its filtration position, and expanding the filter to its filtration position, may occur at different points in the overall sequence. For instance, the step of inserting the filter insertion apparatus may occur before or after the step of administering anesthesia, or before, during or after the surgical procedure itself. Likewise, the step of inserting the filter to its filtration position may occur before or after the step of administering anesthesia, or before, during or after the surgical procedure itself. The same applies for the step of expanding the filter to its filtration position, although preferably this occurs shortly before any restriction in blood flow is released, e.g., just prior to or upon completion of the surgical procedure. Several examples, with relevant comments, of different sequencing alternatives, are provided below.

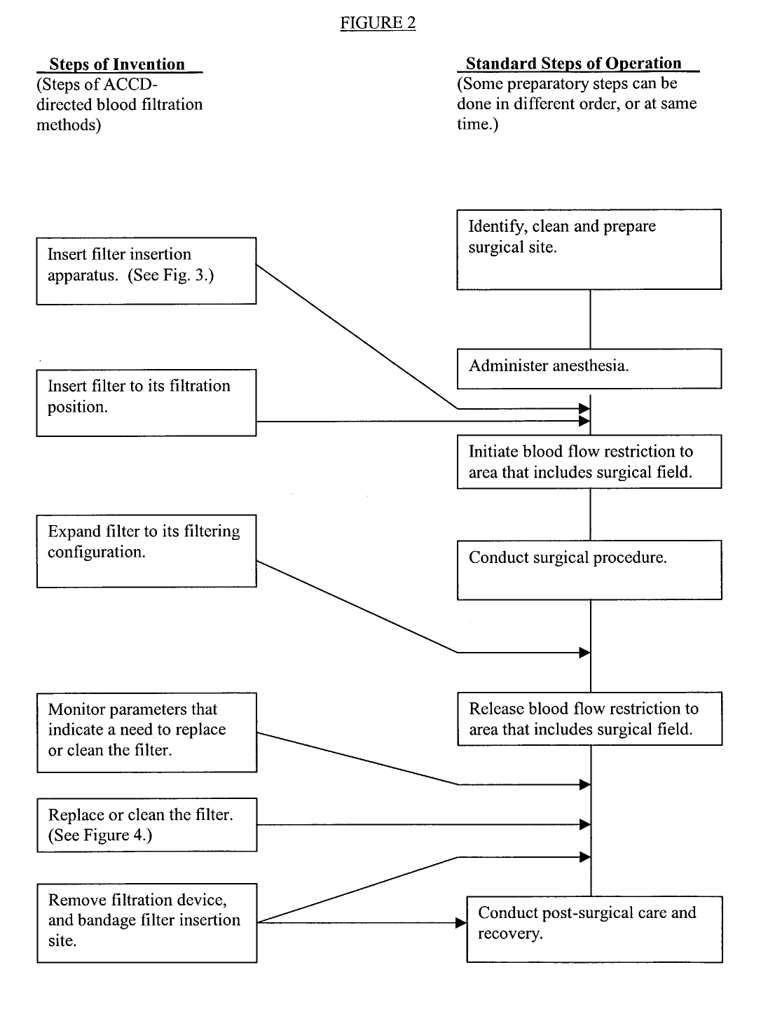

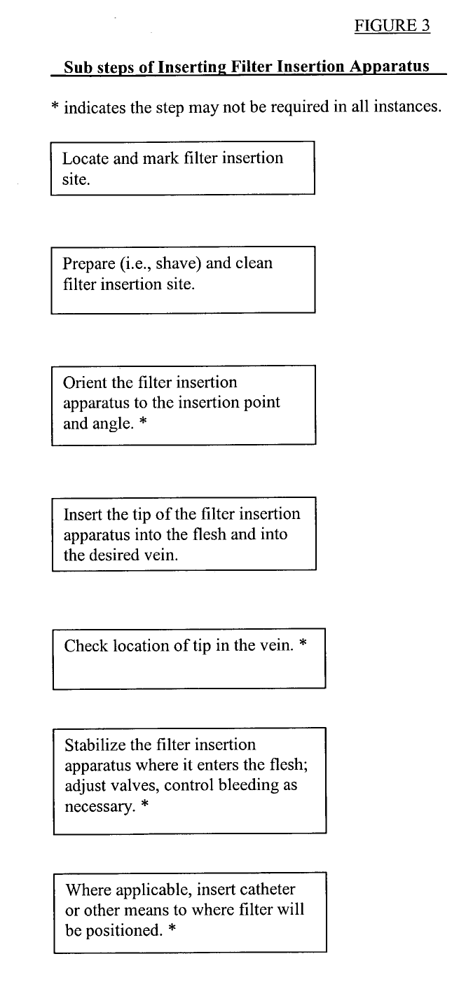

Thus, in one embodiment of the invention, depicted in FIG. 2, an orthopedic surgeon prepares a patient for knee replacement surgery. The step of inserting the filter insertion apparatus is done after the step of identifying, cleaning and preparing the surgical site itself, after the administration of anesthesia, and prior to the initiation of a restriction of blood flow to the area that includes the surgical field. It is between the latter two standard operation steps that the sub steps, as depicted in FIG. 3, of the step of inserting the filter insertion apparatus are conducted. These sub steps include, but are not limited to: locating and marking the site; cleaning the site; orienting the filter insertion apparatus to the position and location to enter the desired vein; and the actual insertion through the flesh to reach and enter the desired vein with the filter insertion apparatus. An optional sub step, depending on the circumstances, is using an imaging system (ultrasound) with a monitor for visualizing the vein to locate the vein and/or to track the passage of the forward end of the filter insertion apparatus to and into the vein. Also, as needed depending on the technician or physician, the apparatus, the patient, and other determinants, the filter insertion apparatus may need to be stabilized at the point where it enters the patient. Depending on the type of filter insertion apparatus, a catheter may be inserted during this step. Typically it is a catheter through which will pass the filter and its tether, guide wires, or other linkage and control mechanisms.

In a simple embodiment, the filter insertion apparatus itself is a needle-pointed cannula through which a catheter that will contain the filter can be passed. The cannula typically has a clamp, valve, or other shut-off control to restrict blood from flowing from the outside-oriented orifice (also referred to as the hub or end of the cannula when this orifice is not closed by a plug or cap, or occupied by a coupling through which the filter tether or guide passes.

In other embodiments, the filter insertion apparatus itself is the catheter that will contain the filter. Here, a simple incision may be made in the skin, and the inward tip of catheter is guided to the desired position, preferably with an imaging capability. Typically, toward the outward end of the catheter is a winged or flattened portion for securing to the body adjacent the incision, a clamp or other shut-off control, and an adaptor, or hub, to receive the filter on its guide wire or tether. In yet other embodiments, a catheter is inserted into a separate orifice-containing housing that is positioned in the flesh and typically extends to the vein (such as a cannula with a wide bore needle at its inward end).

Further, it is noted that the orientation of the filter and the cannula, tether, and/or control mechanisms may vary, and this will affect the relative juxtaposition of the entry point on the body of the filter insertion apparatus and the point in the vein, within the body, where the filter is ultimately deployed. For instance, in U.S. Pat. Nos. 4,873,978 (issued Oct. 17, 1989) and 6,231,589 B1 (issued May 15, 2001), the mechanisms to deliver, deploy, and retrieve the filters are positioned “upstream” of the filter when deployed. That is, the flowing blood encounters the filter before it encounters the cannula, tether, etc. In contrast, in U.S. Pat. No. 6,270,513 B1 (issued Aug. 7, 2001), the cannula delivering and suspending the filter is positioned “downstream” of the deployed filter. Either arrangement, or juxtaposition, of the filter in relation to the mechanisms to deliver, deploy, and retrieve the filter, can be utilized in the present invention.

This leads to the fact that a filter used to capture emboli from a surgical procedure taking place on a limb need not be positioned in that limb. Likewise, the entry point of the filter insertion apparatus need not be positioned on that limb. That is, in certain embodiments the filter is within the trunk of the body, typically in a vein from the limb having surgery, but at a point before merging with a major vein draining another body area. In other embodiments, the filter is introduced into a vein in the lower extremity opposite from the one undergoing the surgical procedure and the catheter and/or filter is/are introduced into a vein draining from the leg undergoing the surgical procedure.

Also, other, more complex filter insertion apparatuses, now known or later developed, may likewise be utilized in this method. Whatever the apparatus used, once the filter insertion apparatus is properly inserted, and the external part of it is stabilized, such as by taping or otherwise securing it in place against the body, this latter stabilizing subtask typically is completed. Normally, change-out of a filter, such as when the filter in use is sufficiently full and requires cleaning or substitution, will take place through and using the same external part of the filter insertion apparatus first established at the entry point in the body.

In certain embodiments, the filter itself is packaged in the same sterile packaging as the filter insertion apparatus. In other embodiments, however, these are packaged separately and the filter itself, on its tether or guide, is prepared for insertion into a receiving orifice on the filter insertion apparatus. The timing of the insertion of the filter itself into the filter insertion apparatus will depend, at a minimum, on the specific recommendations for use of these components together, the circumstances of the surgery and the patient's condition, and the surgeon's preference.

As depicted in FIG. 2, after the step of inserting the filter insertion apparatus into its position, the filter is inserted into its position for filtration in the vein. This is done, typically, by sliding the filter, attached at or near one end of a guide wire or tether, through the filter insertion apparatus (which may be or include a catheter, per above) to the desired position in the vein. Depending on the style of the filter, and its intended means of opening, closing, and retaining filtered emboli while being retrieved, a second control wire or tether, or other means to control the expansion and positioning of the filter, may be provided.

Also as depicted in FIG. 2, after the step of conducting the surgical procedure, and preferably before the step of releasing the blood flow restriction to the area that includes the surgical field, the filter is expanded to its filtering configuration. Depending on the nature of the surgery, including the duration of the restriction of blood flow (if such occurred), the filter may fill with emboli and need to be replaced. The need for this can be assessed by monitoring the pressure proximal to the filter or the differential in pressures across the filter to determine when the filter should be removed and exchanged for a clean filter. Alternately, the need for such change-out of filters can be assessed by ultrasound evaluation of the filter itself, optionally aided by an injected sonically opaque material that binds to emboli, aiding in the visualization of emboli in the filter. These two examples are not meant to be limiting, and other ways to detect the fullness of the filter, now known or later developed, may be utilized.

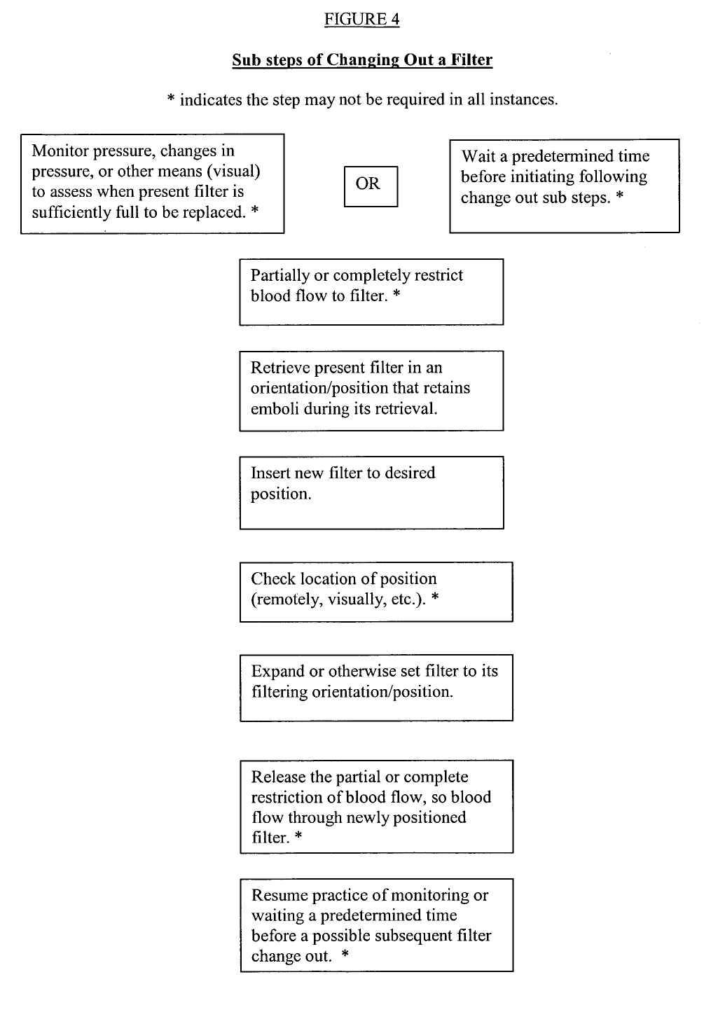

FIG. 4 presents typical sub steps used to change out or clean a filter. The precise steps to change out a filter will depend on the type of filter and its control and retrieval mechanisms. Preferably, a partial or complete restriction of blood flow through the vein is effectuated during this change out, to prevent emboli from passing through the area while no filter is in place. Then, once the old filter has been retrieved and a new filter is in place and expanded to its filtering position, the blood flow restriction is removed to allow the return of blood flow through the filter. The monitoring and change-outs of filters continue for a period determined to remove most or all (statistically) of the surgically-related emboli. It is noted that instead of actual replacement of a filter, the original filter may be cleaned of emboli by appropriate rinsing out or cleaning of the emboli with sterile solutions, or by other means known to those skilled in the art, and thereafter the same filter, now clean, is re-inserted into place and opened to filter again.

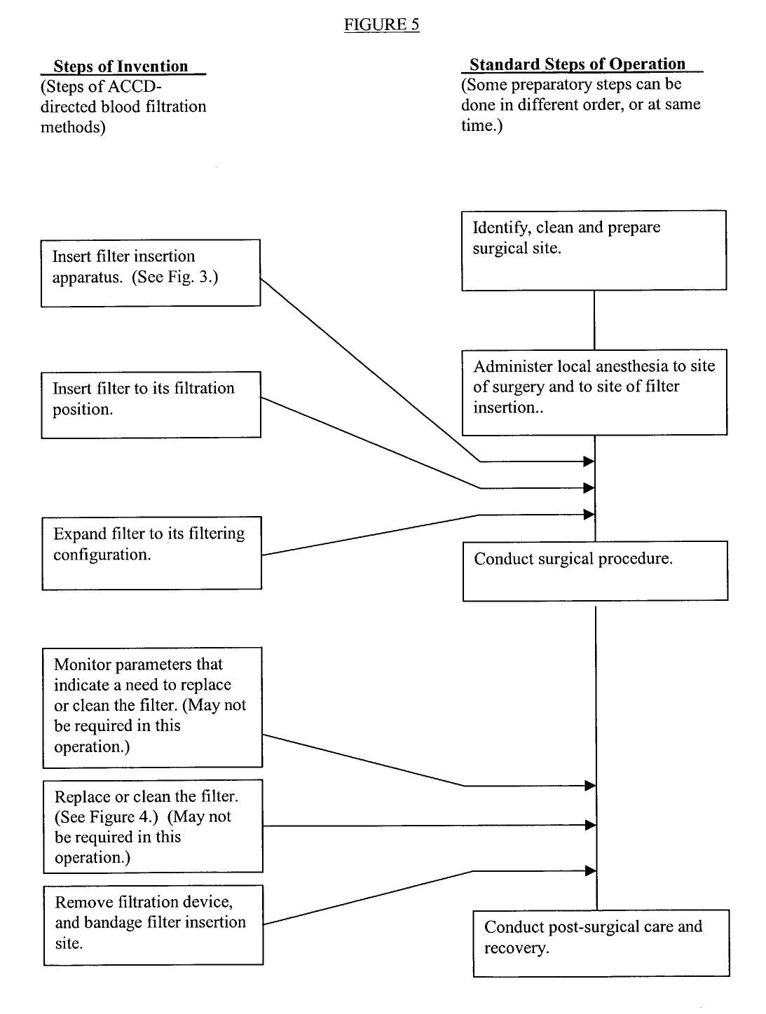

In another embodiment of the invention, the steps of which are provided in FIG. 5, depicts the method as applied to an orthopedic surgery on a patient for a relatively simple left elbow repair. Here, only local anesthesia is applied, both to the surgical site and a lesser amount to the area of insertion of the filter insertion apparatus. The filter is inserted and deployed (expanded to full size to optimally capture emboli) prior to beginning the surgery, and the blood flow to the arm is not restricted during the entire surgical procedure. In this case, the filter is designed to allow the free flow of blood and to only trap emboli of a size larger than normal blood elements. However, should there be a need to change out the filter, as discussed above, it is preferable that the blood flow be temporarily restricted, even if partially and not fully.

Further, in comparing the steps in the right columns of FIGS. 2 and 5, it is noted that the standard steps of the operation depicted in FIG. 5 do not include the steps of “initiate blood flow restriction to area that includes surgical field” and “release blood flow restriction to area that includes surgical field.” This is to indicate that the present invention, in certain embodiments, serves the purpose of reducing or eliminating post-surgical cognitive dysfunction even when the particular surgical procedure does not include these two steps. In surgical procedures that do not involve blood flow restriction, assuming other factors are roughly equivalent, fewer emboli are expected to be released at a particular stage in the surgical procedure, consequently the emboli “shower” over a more protracted period of time and may be less likely to traverse the lung. As a result, other factors being roughly equivalent, a less intensive filtration effort is expected to be required where there is no blood flow restriction.

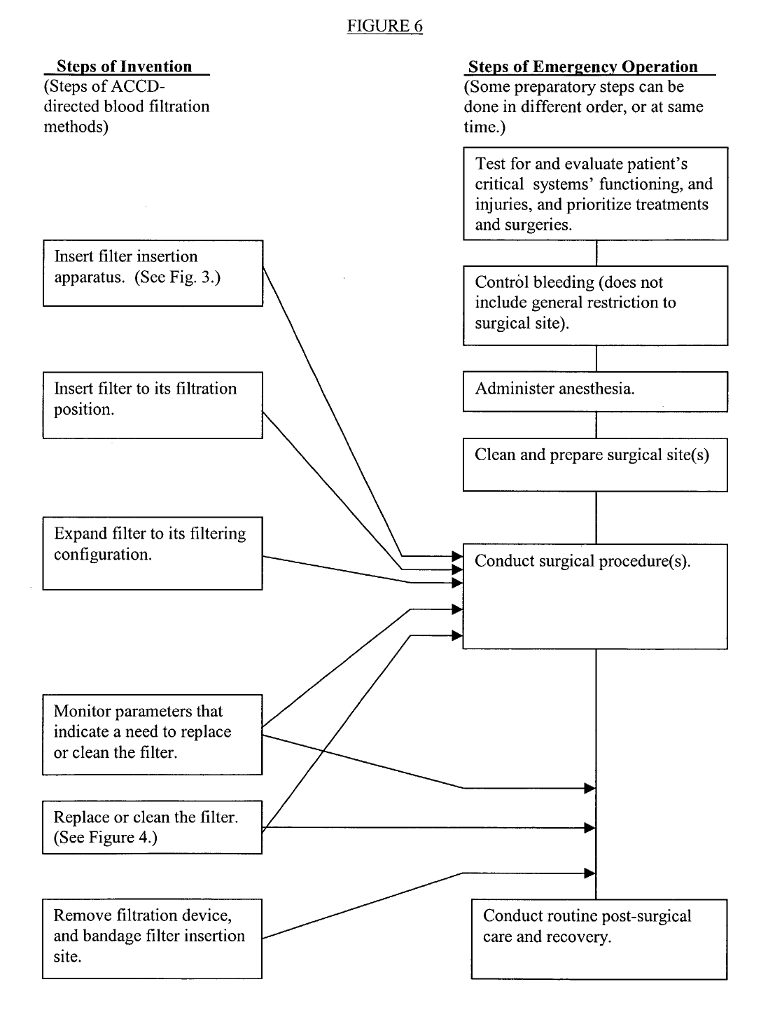

FIG. 6 depicts an embodiment of the methods of the present invention in which a blood filtration procedure is conducted during an emergency surgery that includes operating on a complex fracture with lacerations to one leg, resulting from a pedestrian/automobile accident. The patient is first evaluated in the emergency room to determine the status of critical functions, such as breathing, blood loss, blood pressure, level of shock, head injuries, heart condition, etc. As this is being assessed, bleeding from the leg wound is being controlled. However, this involves direct pressure and not a general restriction to the wound site.

In immediate preparation for surgery, antiseptic is provided for the surgical procedure itself. Additional cleaning and preparation of the wound area may be done at this time. Then the surgery is initiated. During this time ruptured vessels are repaired. Also during this time a team conducts the steps of inserting the filter insertion apparatus, inserting the filter to its filtration position (in a vein returning blood from the leg wound), and expanding the filter to its filtering configuration. These steps have been delayed given the emergency status of the patient. However, once the patient's condition is assessed and the critical medical issues are being attended to, and staff is available, these steps to filter blood to reduce and/or eliminate post-surgical cognitive dysfunction are effectuated. Also, as appropriate, parameters are monitored that may indicate the need to replace or clean the filter, and the filter is replaced or cleaned by the sub steps indicated previously. The filter may be kept in place for a sufficient period after surgery to assure capture of emboli as the trauma site inflammation lessens. For this reason, two lines are shown from each of the last-noted steps.

It is known that there are different surgical approaches to achieve the same operative objective. For instance, in total knee replacement surgeries, there are the anteromedial, midline, and anterolateral incision approaches. Each has its own rationale and supporters. Given such types of operative variation among surgeons and surgical facilities, it is likely that sequencing variation in the steps described herein will be quite common. However, for this and other orthopedic surgeries of the extremities, and for other types of surgeries in the extremities that are expected to result in potentially damaging amounts of released emboli, the method of filtering the return blood from the limb to reduce or eliminate cognitive dysfunction is appropriate to consider and implement.

The following example further indicates the utility and benefit of the invention methods.

### EXAMPLE 1

In laboratory experiments performed with swine, total knee prostheses were inserted bilaterally after placement of tourniquets. The tourniquets were maintained in place for two hours, approximately the time they are applied during total knee replacements in humans. Doppler flow probes were placed on the carotid arteries bilaterally and monitored for emboli traversing the lungs and entering the vessels leading to the brain. Small numbers of emboli were noted prior to release of the tourniquets and large “showers” of emboli were noted after tourniquet release, particularly after release of the second tourniquet, indicating that the large number of emboli released overwhelmed the normal defensive filtering by the lung. Further, if a vascular clamp was placed around the large vein draining blood from the lower extremity prior to release of the tourniquet, the emboli were not detected. When the clamp was removed, the emboli were detected in the carotids, indicating that a filter would capture the emboli released and prevent cerebral involvement.

All patents, patent applications, publications, texts and references discussed or cited herein are incorporated by reference to the same extent as if each individual publication or patent application was specifically and individually set forth in its entirety. Nothing herein is to be construed as an admission that the invention is not entitled to antedate such disclosures by virtue of prior invention. In addition, all terms not specifically defined are first taken to have the meaning given through usage in this disclosure, and if no such meaning is inferable, their normal meaning. Where a limitation is described but not given a specific term, a term corresponding to such limitation may be taken from any references, patents, applications, and other documents cited herein, or, for an application claiming priority to this application, additionally from an Invention Disclosure Statement, Examiner's Summary of Cited References, or a paper otherwise entered into the file history of this application.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. Thus, for the above variations and in other regards, it should be understood that the examples and embodiments described herein are for illustrative purposes only and that various modifications or changes in light thereof will be suggested to persons skilled in the art and are to be included within the spirit and purview of this application and the scope of the appended claims.

Also, although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included in the scope of this invention as defined in the following claims. In the claims, means-plus-function clauses and step-plus-function clauses are intended to cover the structures described herein as effectuating or performing the recited function and to cover not only structural equivalents, but also to cover equivalent structures as one of ordinary skill in the art would understand equivalence with regard to a any means or any step that will achieve a stated function in an equivalent manner. For instance, a “means to filter emboli in a vessel” should be taken to include methods now or later known to those of skill in the art regardless of differences in the exact steps, materials and combinations of structures required to achieve this function.

What is claimed is:

1. A method for reducing or preventing post-surgical cognitive dysfunction in a patient undergoing a surgical procedure, comprising:
a. inserting an expandable temporary filter into a vein leading from a site of said surgical procedure;
b. expanding said temporary filter at a point in said vein to catch emboli; and
c. removing said filter to remove said emboli,
wherein the removing of said emboli in said filter reduces or prevents the occurrence of post-surgical cognitive dysfunction.

2. The method of claim 1 wherein said surgical procedure is on a limb of said patient.

3. A method for reducing or preventing post-surgical cognitive dysfunction in a patient undergoing a surgical procedure, comprising:
a. inserting an expandable temporary filter into a vein leading from a site of said surgical procedure;
b. initiating a restricting of blood flow to said site during said surgical procedure;
c. expanding said temporary filter at a point in said vein to catch emboli;
d. ending said restricting of blood flow, wherein blood retained due to said restricting, containing a quantity of emboli, passes through said filter in said vein and emboli are captured by said filter, and
e. removing said filter to remove said emboli,
wherein the removing of said emboli in said filter reduces or prevents the occurrence of post-surgical cognitive dysfunction.

4. The method of claim 3, wherein said restricting is applying a tourniquet to a limb proximal to the site of surgery.

5. The method of claim 4, wherein said surgical procedure is an orthopedic surgical procedure.

6. The method of claim 4, wherein said surgical procedure is a knee replacement.

7. The method of claim 4, wherein said inserting is into the iliac or the femoral vein.
